(19) United States
(12) Patent Application Publication
(10) Pub. No.: US 2011/0184185 A1
(43) Pub. Date: Jul. 28, 2011

(54) Methods Of Preparing Thiazolidines

(75) Inventors: Herbert T. Nagasawa, Irvine, CA (US); Kurt Wachholder, Gilberts, IL (US)

(73) Assignee: MAX INTERNATIONAL, LLC, Midvale, UT (US)

(21) Appl. No.: 13/015,941

(22) Filed: Jan. 28, 2011

Related U.S. Application Data
(60) Provisional application No. 61/299,040, filed on Jan. 28, 2010.

(51) Int. Cl. C07D 277/06

(52) U.S. Cl. 548/201

(57) ABSTRACT

The present invention provides methods of preparing thiazolidines, and in particular methods of preparing ribose-cysteine.

### CROSS-REFERENCE TO RELATED APPLICATIONS

This application claims priority to U.S. Provisional Application No. 61/299,040, filed Jan. 28, 2010, of which is hereby incorporated by reference in its entirety.

### FIELD OF THE INVENTION

The present invention is directed, in part, to methods of preparing thiazolidines and, in particular, to methods of preparing ribose-cysteine.

### BACKGROUND OF THE INVENTION

Several methods have been used to prepare thiazolidine compounds (see, Yan et al, High Technology Lett., 2005, 11, 437-442; Bognar et al, Liebigs Ann. Chem, 1970, 738, 68-78; Roberts et al, J. Med. Chem., 1987, 30, 1891; Roberts et al., Med. Chem. Res., 1991, 1, 213-219; Roberts et al., Anticancer Res., 1994, 14, 383-387; Roberts et al., Radiation Res., 1995, 143, 203-213; and Weitzel et al., Hoppe Seylers Z Physiol. Chem., 1959, 315, 236-55, each of which is incorporated herein by reference in its entirety). There is still a need, however, for improved methods of preparing thiazolidine compounds that can yield stable and/or pure thiazolidine compounds in a cost efficient manner. The present invention fulfills these needs as well as others.

### SUMMARY OF THE INVENTION

In some embodiments, the present invention provides methods of preparing a thiazolidine comprising contacting a sugar and a cysteine with an aqueous solution under suitable conditions to form the thiazolidine; and isolating the thiazolidine.

In some embodiments, the present invention provides methods of preparing a thiazolidine comprising contacting an aldose or ketose monosaccharide or an amino sugar, and a cysteine with an aqueous solution under conditions to form the thiazolidine; precipitating the thiazolidine; and vacuum drying the thiazolidine at a temperature that is less than or equal to 65° C., wherein the aqueous solution optionally comprises ethanol.

In some embodiments, the present invention provides methods of preparing a thiazolidine comprising in the following order: a) contacting an aldose or ketose monosaccharide or an amino sugar with an aqueous solution; contacting the solution of step a) with a cysteine and ethanol; c) precipitating the solution of step b) to yield a thiazolidine precipitate; and d) vacuum drying the thiazolidine precipitate. In some embodiments, step b) comprises in the following order: i) contacting the solution of step a) with the cysteine; and ii) contacting the solution of step i) with the ethanol.

### DESCRIPTION OF EMBODIMENTS

Throughout the present specification, various sugars, amino acids, and other molecules that have both D- and L-forms are disclosed. Unless explicitly stated otherwise, recitation of the sugar, amino acid, or other molecule can refer to the D-form, L-form, or a mixture of both. In some embodiments, the sugar, amino acid, or other molecule will be free of the D-form, i.e. 100% L-form. Likewise, in some embodiments, the sugar, amino acid, or other molecule will be free of the L-form. For example, the term “ribose” can refer to D-ribose, L-ribose, or a mixture of both. Additionally, the term “cysteine” refers to L-cysteine, D-cysteine, or a mixture of both. These are non-limiting examples and other molecules referred to herein can also have D- and L-forms, which are also included within the presently described inventions.

As used herein, the singular forms “a,” “an,” and “the” include plural references unless the context clearly dictates otherwise.

As used herein, the term “about” means ±5% of the value being modified.

As used herein, the phrase “sugar-cysteine product” refers to a product that forms when a sugar and cysteine react with one another. Examples of sugar-cysteine products include, but are not limited to, ribose-cysteine (RibCys), glucose-cysteine (GlcCys), fructose-cysteine (FruCys), glyceraldehyde-cysteine (GlycCys), glucosamine-cysteine (GlcNH₂Cys), and N-acetylglucosamine-cysteine (GlcNHAcCys), and the like, and/or any combination thereof.

As used herein, the term “sugar” refers to a saccharide. The saccharide can be either a polysaccharide or a monosaccharide. In some embodiments, the monosaccharide is an aldose or ketose monosaccharide. Monosaccharides include, but are not limited to, fructose, glucose, ribose, and the like. In some embodiments, the sugar is mannose, arabinose, xylose, rhamnose, lyxose, galactose, or the like. The sugar can also be an amino sugar. Examples of amino sugars include, but are not limited to, N-acetylglucosamine, galactosamine, glucosamine, and the like. In some embodiments, the compositions and methods described herein are free of glucose or are at least free of detectable glucose. In some embodiments, the compositions and methods described herein are free of fructose or are at least free of detectable fructose. The compositions can also be free of sucrose or are at least free of detectable sucrose. In some embodiments, the compositions and methods described herein are free of all sugars or are at least free of any detectable sugars, except for the sugar forming the sugar-cysteine product.

As used herein, the term “ratio” refers to the amounts of two or more compounds, molecules, or the like, compared to one another. The ratio can be, for example, in terms of absolute weight (e.g., grams to grams). The ratio can be also be, for example, determined by comparing concentrations of each compound (e.g., molarity to molarity). The ratio can also be in terms of moles of each molecule present in the composition. For example, a composition comprising a first and second compound each with 10 mmol would be said to be in a 1 to 1 ratio (i.e., 1.0:1.0).

As used herein, the term “substantially” means at least 95%, at least 96%, at least 97%, at least 98%, or at least 99%.

As used herein, the term “RibCys” refers to 2(R,S)-D-ribo-(1′,2′,3′,4′-tetrahydroxybutyl)thiazolidine-4(R)-carboxylic acid. The chemical name can also be referred to as “(4R)-2-(1,2,3,4-tetrahydroxybutyl)thiazolidine-4-carboxylic acid.” RibCys can be represented by the following formula I:

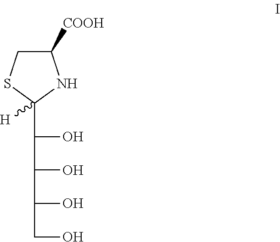

As used herein, the term “GlcCys” refers to the product of glucose and cysteine. “GlcCys” can be represented by the following formula II:

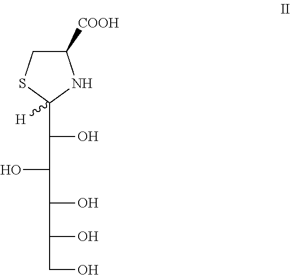

The present invention provides methods of preparing thiazolidine compounds. One type of thiazolidine compound is a sugar-cysteine product. The present invention, in some embodiments, comprises contacting a sugar and a cysteine with an aqueous solution under conditions to form the thiazolidine. In some embodiments, the thiazolidine compound is isolated.

In some embodiments, the cysteine is L-cysteine. In some embodiments, the cysteine is a cysteine salt. For example, the cysteine salt can be, but is not limited to, a cysteine hydrochloride salt. In some embodiments, the cysteine is a salt monohydrate. In some embodiments, the composition used in the methods described herein is free of a cysteine salt. In some embodiments, the composition used in the methods described herein is free of a cysteine salt monohydrate. In some embodiments, the cysteine is the free base form of cysteine. In some embodiments, the methods described herein employ a composition comprising cysteine that is at least 98% pure. In some embodiments, the composition comprising cysteine should be substantially free of cystine. In some embodiments, the cysteine is free of cystine. In some embodiments, the cysteine comprises less than 2%, less than 1%, less than 0.5%, less than 0.4%, less than 0.3%, less than 0.2%, less than 0.1%, or less than 0.5% cystine. The percentage cystine present in the cysteine composition can be determined by comparing w/w, mol/mol, ppm/ppm, and the like.

In some embodiments, the present invention provides methods of preparing a thiazolidine comprising: 1) contacting a sugar with an aqueous solution; and 2) contacting a cysteine with the aqueous solution comprising the sugar, wherein the sugar and cysteine are contacted under conditions sufficient to form a thiazolidine. In some embodiments, the cysteine is contacted with the solution comprising the sugar after the sugar is completely dissolved. In some embodiments, the sugar is contacted with the aqueous solution before a cysteine is contacted with the aqueous solution.

In some embodiments, the present invention provide methods of preparing a thiazolidine comprising: 1) contacting a cysteine with an aqueous solution; and 2) contacting a sugar with the aqueous solution comprising the cysteine, wherein the sugar and cysteine are contacted under conditions sufficient to form the thiazolidine.

In some embodiments, the aqueous solution is water. In some embodiments, the water is USP purified water. In some embodiments, the aqueous solution comprises an alcohol. The alcohol can be, for example, ethanol, methanol, isoproponal, denatured ethanol, such as, but not limited to SDA-3A ethanol and the like. In some embodiments, the aqueous solutions described herein comprise water and alcohol. In some embodiments, the ratio of water to alcohol is about 1:1, about 2:1, about 4:1, about 5:1, about 10:1, about 20:1, about 25:1, about 50:1, about 75:1, about 90:1, about 100:1, about 500:1, about 1000:1, or about 10000:1 (v/v). In some embodiments, the ratio of the sugar to the cysteine is 1.0:1.0. In some embodiments, the ratio is greater than 1.0:1.0. For example, the ratio of sugar to cysteine can be about 1.1:1.0, about 1.5:1.0, about 2.0:1.0, about 3.0:1.0, about 4.0:1.0, about 5.0:1.0, about 6.0:1.0, about 7.0:1.0, about 8.0:1.0, about 9.0:1.0, or about 10.0:1.0, and the like. In some embodiments, the ratio of the sugar to cysteine is from about 2.0:1.0 to about 10.0:1.0. In some embodiments, the ratio of the sugar to cysteine is from about 2.0:1.0 to about 5.0:1.0. In some embodiments, the ratio of the sugar to cysteine is greater than about 2.0:1.0, greater than about 3.0:1.0, greater than about 4.0:1.0, greater than about 5.0:1.0, greater than about 6.0:1.0, greater than about 7.0:1.0, greater than about 8.0:1.0, greater than about 9.0:1.0, or greater than about 10.0:1. In some embodiments, the ratio determined mol:mol. In some embodiments, the pH of the solution is less than or equal to about 7.5. In some embodiments, the pH is from about 4.0 to about 7.5, from about 4.0 to about 7.0, from about 4.0 to about 6.5, from about 4.0 to about 6.0, from about 4.0 to about 5.5, from about 4.0 to about 5.0, from about 4.5 to about 5.0, from about 6.0 to about 7.5, from about 6.0 to about 7.0, from about 6.0 to about 6.5, from about 6.5 to about 7.5, from about 6.8 to about 7.2, from about 6.9 to about 7.1, or from about 7.0 to about 7.5. In some embodiments, the pH is about 7.0.

In some embodiments, the present invention provides methods of preparing a thiazolidine comprising contacting a sugar, a cysteine, and an alcohol with an aqueous solution under conditions sufficient to form the thiazolidine. In some embodiments, the sugar is contacted with an aqueous solution that is free of cysteine. In some embodiments, the sugar is contacted with an aqueous solution that is free of an alcohol. In some embodiments, the sugar is contacted with an aqueous solution that is free of an alcohol and a cysteine. In some embodiments, the cysteine and/or the alcohol are contacted with an aqueous solution comprising the sugar after the sugar is completely dissolved. In some embodiments, the alcohol is contacted with the solution prior to the cysteine being contacted with the solution comprising the sugar. In some embodiments, the cysteine is contacted prior to the alcohol being contacted with the solution comprising the sugar. In some embodiments, the alcohol is contacted prior to the cysteine being contacted with the solution comprising the sugar. In some embodiments, a cysteine and an alcohol are contacted with a solution comprising a sugar simultaneously.

The solution comprising the cysteine and the sugar can be mixed for any period of time sufficient to form the thiazolidine. For example, the solution can be mixed for at least 2 hours, for at least 4 hours, for at least 6 hours, for at least 8 hours, for at least 12 hours, for at least 16 hours, for at least 20 hours, or for at least 24 hours. The solution can also be mixed for about 2 hours, for about 4 hours, for about 6 hours, for about 8 hours, for about 10 hours, for about 12 hours, for about 14 hours, for about 16 hours, for about 18 hours, for about 20 hours, for about 22 hours, or for about 24 hours. In some embodiments, the solution can be mixed for less time.

In some embodiments, the solution is mixed at a temperature from about 20° C. to about 25° C. The solution can also be mixed at a temperature from about 32° C. to about 37° C. In some embodiments, the solution can be mixed at a temperature that is less than about 10° C., less than about 9° C., less than about 8° C., less than about 7° C., less than about 6° C., less than about 5° C., less than about 4° C., less than about 3° C., less than about 2° C., or less than about 1° C.

In some embodiments, the ratio of cysteine to alcohol is about or at least 2:1, about or at least 4:1, about or at least 6:1, about or at least 8:1, or about or at least 10:1 (w:w).

In some embodiments, the methods of preparing a thiazolidine comprise precipitating the thiazolidine. Precipitation can be performed by any method sufficient to precipitate the thiazolidine out of solution. In some embodiments, precipitating the thiazolidine comprises contacting the thiazolidine with a second amount of an alcohol. In some embodiments, the alcohol used to precipitate the thiazolidine is the same alcohol that is used to synthesize the thiazolidine as described herein. In some embodiments, the alcohol is a different alcohol. In some embodiments, the alcohol used to precipitate the thiazolidine is methanol, isopropanol, ethanol, denatured ethanol (e.g. SDA-3A ethanol) and/or any combination thereof. In some embodiments, precipitating the thiazolidine can comprise mixing the solution comprising the thiazolidine at a temperature less than or equal to about 10° C., less than or equal to about 9° C., less than or equal to about 8° C., less than or equal to about 7° C., less than or equal to about 6° C., less than or equal to about 5° C., less than or equal to about 4° C., less than or equal to about 3° C., less than or equal to about 2° C., or less than or equal to about 1° C. In some embodiments, precipitating the thiazolidine can comprise mixing the solution comprising the thiazolidine at a temperature from about 0° C. to about 10° C., about 0° C. to about 6° C., or about 0° C. to about 5° C.

In some embodiments, the amount of alcohol contacted with the thiazolidine to precipitate the thiazolidine is in a ratio of about or at least 8.0:1.0 (alcohol:cysteine; w/w). In some embodiments, the amount of alcohol contacted with the thiazolidine to precipitate the thiazolidine is in a ratio of at least 7.0:1.0, at least 8.0:1.0, at least 9.0:1.0, or at least 10.0:1.0 (alcohol:cysteine; w/w). In some embodiments, the thiazolidine is precipitated for at least 2 hours, at least 3 hours, at least 4 hours, at least 5 hours, at least 6 hours, at least 7 hours, at least 8 hours, at least 9 hours, or at least 10 hours. In some embodiments, the thiazolidine is precipitated for about 2 hours, about 3 hours, about 4 hours, about 5 hours, about 6 hours, about 7 hours, about 8 hours, about 9 hours, or about 10 hours.

In some embodiments, the thiazolidine is precipitated at a first temperature for a first period of time and then a second temperature for a second period of time, wherein the second temperature is less than the first temperature. In some embodiments, the thiazolidine is precipitated (e.g., by the addition of ethanol) at a first temperature that is from about 18° C. to 30° C. and then a second temperature that is less than 10° C. In some embodiments, the second temperature can be less than about 10° C., less than about 9° C., less than about 8° C., less than about 7° C., less than about 6° C., less than about 5° C., less than about 4° C., less than about 3° C., less than about 2° C., less than about 1° C., or 0° C. In some embodiments, the second temperature can be from about 0° C. to about 10° C., from about 0° C. to about 6° C., or from about 0° C. to about 5° C. In some embodiments, the first temperature can be, for example, from about 18° C. to 30° C., from about 18° C. to 25° C., from about 20° C. to 27° C., or from about 20° C. to 25° C. In some embodiments, the first temperature is about 18° C., about 19° C., about 20° C., about 21° C., about 22° C., about 23° C., about 24° C., about 25° C., about 26° C., about 27° C., about 28° C., about 29° C., or about 30° C. In some embodiments, the thiazolidine is precipitated at a first temperature for at least 2 hours, at least 3 hours, at least 4 hours, at least 5 hours, at least 6 hours, at least 7 hours, at least 8 hours, at least 9 hours, or at least 10 hours. In some embodiments, the thiazolidine is precipitated at a first temperature for about 2 hours, for about 3 hours, for about 4 hours, for about 5 hours, for about 6 hours, for about 7 hours, for about 8 hours, for about 9 hours, or for about 10 hours. In some embodiments, the thiazolidine is precipitated at a second temperature for at least 2 hours, at least 3 hours, at least 4 hours, at least 5 hours, at least 6 hours, at least 7 hours, at least 8 hours, at least 9 hours, or at least 10 hours. In some embodiments, the thiazolidine is precipitated at a second temperature for about 2 hours, about 3 hours, about 4 hours, about 5 hours, about 6 hours, about 7 hours, about 8 hours, about 9 hours, or about 10 hours.

In some embodiments, the present invention provides methods wherein the thiazolidine is collected by filtration. In some embodiments, the thiazolidine that is filtered is precipitated thiazolidine. In some embodiments, the thiazolidine is collected by centrifugation. In some embodiments, the thiazolidine is washed with a washing agent.

As used herein, the phrase “washing agent” refers to an agent that can be used to wash away or solubilize impurities present in a composition comprising a thiazolidine. In some embodiments, the washing agent is an agent in which thiazolidine is not soluble. In some embodiments, the thiazolidine is completely insoluble in the washing agent. In some embodiments, the washing agent is an alcohol. Examples of washing agents include, but are not limited to, ethanol, methanol, isopropanol, denatured ethanol (e.g. SDA-3A ethanol) and the like.

In some embodiments, the present invention provides methods of preparing a thiazolidine monohydrate. The methods described herein can be used to prepare a thiazolidine monohydrate. In some embodiments, the present invention provides methods of preparing an anhydrous thiazolidine. The methods described herein can be used to prepare an anhydrous thiazolidine.

In some embodiments, the methods for preparing thiazolidines comprise drying the thiazolidine. In some embodiments, the thiazolidine is dried for a sufficient period of time such that the thiazolidine is dried to become an anhydrous thiazolidine. Any drying method can be used to dry the thiazolidine sufficiently. In some embodiments, a drying method is used to remove the water and/or alcohol from a composition comprising a thiazolidine. An example of a drying technique includes, but is not limited to, drying the thiazolidine under vacuum. In some embodiments, the thiazolidine is dried at a temperature that is less than about 65° C., less than about 60° C., less than about 55° C., less than about 50° C., less than about 45° C., less than about 40° C., less than about 35° C., less than about 30° C., or less than about 20° C. In some embodiments, the thiazolidine is dried at a temperature from about 20° C. to 65° C., from about 30° C. to 65° C., from about 40° C. to 65° C., from about 50° C. to 65° C., from about 60° C. to 65° C., from about 20° C. to 50° C., from about 30° C. to 50° C., from about 40° C. to 50° C., from about 45° C. to 50° C., from about 20° C. to 40° C., from about 30° C. to 40° C., from about 35° C. to 40° C., from about 20° C. to 30° C., from about 25° C. to 30° C., or from about 20° C. to 25° C. The thiazolidine can also be dried under vacuum at the various temperatures described herein.

In some embodiments, the methods of preparing a thiazolidine yield at least 90%, at least 91%, at least 92%, at least 93%, at least 94%, at least 95%, at least 96%, or at least 97% of a calculated theoretical yield. The yield can be determined by using the amounts of the starting materials. Methods of calculating the yield are known to one of skill in the art.

In some embodiments, the present invention provides methods of preparing a thiazolidine that is at least 95% pure as determined by HPLC. In some embodiments, the present invention provides methods of preparing a thiazolidine that comprises less than 100, 120, 130, 140, 150, 160, 170, 180, 190, 200, 210, 220, 225, 250, 300, 400, or 500 ppm of an alcohol. The alcohol may be any alcohol including, but not limited to, methanol, ethanol isopropanol, denatured ethanol (e.g. SDA-3A ethanol) and the like.

In some embodiments, the isolated thiazolidine comprises a monohydrate thiazolidine. In some embodiments, the isolated thiazolidine comprises anhydrous thiazolidine. In some embodiments, the isolated thiazolidine is free of a monohydrate thiazolidine.

In some embodiments, the present invention provides methods of preparing a thiazolidine comprising: a) contacting an aldose monosaccharide or an amino sugar, and a cysteine with an aqueous solution under conditions to form the thiazolidine; b) precipitating the thiazolidine; and c) vacuum drying the thiazolidine at a temperature that is less than or equal to 65° C., wherein the aqueous solution optionally comprises ethanol.

In some embodiments, the present invention provides methods of preparing a thiazolidine comprising in the following order: a) contacting an aldose monosaccharide or an amino sugar, with an aqueous solution; b) contacting the solution of step a) with a cysteine and ethanol; c) precipitating the solution of step b) to yield a thiazolidine precipitate; and d) vacuum drying the thiazolidine precipitate. In some embodiments, step b) comprises in the following order: i) contacting the solution of step a) with the cysteine; and ii) contacting the solution of step i) with the ethanol.

In some embodiments, the present invention provides methods of preparing kilogram quantities of thiazolidines. In some embodiments, the methods yield at least 10 kg, at least 15 kg, at least 20 kg, at least 25 kg, at least 50 kg, at least 75 kg, at least 80 kg, at least 85 kg, at least 90 kg, at least 95 kg, or at least 100 kg of a thiazolidine. The methods described herein can also be used to yield a thiazolidine that is at least 95%, at least 96%, at least 97%, at least 98%, or at least 99% pure as measured by HPLC. The methods described herein can also be used to yield a thiazolidine that is 100% pure as measured by HPLC.

The products prepared with the methods disclosed herein can be used in various forms and can be administered in standard manners. The compositions can also be used for different purposes. See, for example, U.S. Patent Application Publication No. 2009-0042822 titled “Use Of Ribose-Cysteine To Treat Hypoxia By Enhancing Delivery Of Glutathione And ATP Levels In Cells,” which is incorporated herein by reference in its entirety.

The compositions described herein (e.g., thiazolidines) can also be prepared according to the methods described herein together with a pharmaceutically-acceptable vehicle, carrier, excipients or diluent. For example, the thiazolidine and the compositions described herein may be administered in a standard manner such as orally, parenterally, transmucosally (e.g., sublingually or via buccal administration), topically, transdermally, rectally, or inhalation (e.g., nasal or deep lung inhalation). Parenteral administration includes, but is not limited to intravenous, intraarterial, intraperitoneal, subcutaneous, and intramuscular.

For buccal administration, the composition can be in the form of a tablet or lozenge formulated in conventional manner. For example, tablets and capsules for oral administration can contain one or more conventional excipients such as binding agents (e.g., syrup, acacia, gelatin, sorbitol, tragacanth, mucilage of starch or polyvinylpyrrolidone), fillers (e.g., lactose, sugar, microcrystalline cellulose, maize-starch, calcium phosphate or sorbitol), lubricants (e.g., magnesium stearate, stearic acid, talc, polyethylene glycol or silica), disintegrants (e.g., potato starch or sodium starch glycollate), or wetting agents (e.g., sodium lauryl sulfate). Tablets may be coated according to methods well known in the art.

Such formulations can also be formulated as suppositories for rectal administration, e.g., containing conventional suppository bases, such as cocoa butter or other glycerides. Compositions for inhalation typically can be provided in the form of a solution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or nonaqueous vehicles, such as eye drops, creams, ointments, lotions, and pastes, or are in the form of a medicated plaster, patch, or membrane.

Additionally, the thiazolidines and compositions described herein can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternately, the active ingredient can be in powder form for constitution with a suitable vehicle (e.g., sterile, pyrogen-free water) before use.

A composition in accordance with the present invention also can be formulated as a depot formulation. Such long acting formulations can be administered by implantation (e.g., subcutaneously or intramuscularly) or by intramuscular injection. Accordingly, the compounds of the invention can be formulated with suitable polymeric or hydrophobic materials (e.g., an emulsion in an acceptable oil), ion exchange resins, or as sparingly soluble derivatives (e.g., a sparingly soluble salt).

For oral administration a composition can take the form of solution(s), suspension(s), tablet(s), pill(s), capsule(s), powder(s), and the like. Tablets containing various excipients such as sodium citrate, calcium carbonate and calcium phosphate are employed along with various disintegrants such as starch, and potato or tapioca starch, and certain complex silicates, together with binding agents such as polyvinylpyrrolidone, sucrose, gelatin and acacia. Additionally, lubricating agents such as magnesium stearate, sodium lauryl sulfate and talc may be used to form tablets. Solid compositions of a similar type are also employed as fillers in soft and hard-filled gelatin capsules. Other materials in this connection also include lactose or milk sugar as well as high molecular weight polyethylene glycols.

Alternately, the compositions described herein can be incorporated into oral liquid formulations such as aqueous or oily suspensions, solutions, emulsions, syrups, or elixirs, for example. Moreover, formulations containing these compounds can be presented as a dry product for constitution with water or other suitable vehicle before use. Such liquid formulations can contain conventional additives, such as suspending agents, such as sorbitol syrup, synthetic and natural gums such as tragacanth, acacia, alginate, dextran, sodium carboxymethylcellulose, methylcellulose, polyvinyl-pyrrolidone or gelatin, glucose/sugar syrup, gelatin, hydroxyethylcellulose, hydroxypropylmethylcellulose, aluminum stearate gel, emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid. The liquid forms in which the compositions described herein may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

When aqueous suspensions and/or elixirs are desired for oral administration, the compounds described herein can be combined with various sweetening agent(s), flavoring agent(s), coloring agent(s), emulsifying agent(s) and/or suspending agent(s), as well as such diluent(s) as water, ethanol, propylene glycol, glycerin and various like combinations thereof. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The compositions described herein can also be administered in a controlled release formulation such as a slow release or a fast release formulation. Such controlled release formulations of the combinations described herein may be prepared using methods well known to those skilled in the art. The method of administration can be determined, by an attendant physician or other person skilled in the art after an evaluation of the patient's condition and/or requirements. The compositions prepared by the methods described herein can also be used for other uses and formulated for different purposes.

In addition to the compositions described herein, the present invention also provides solid form preparations which are intended to be converted, shortly before use, to liquid form preparations for oral administration to an animal (e.g., oral administration). Such liquid form preparations include solutions, suspensions, and emulsions. Particular solid form preparations can be provided in unit dose form. The unit dose form can provide convenience to the user. The unit dose form can be used to provide a single liquid dosage unit. Alternately, sufficient solid form preparations may be provided so that after conversion to liquid form, multiple individual liquid doses may be obtained by measuring predetermined volumes of the liquid form preparation as with a syringe, teaspoon, or other volumetric container or apparatus. In some embodiments, when multiple liquid doses are so prepared, the unused portion of the liquid doses can be kept at low temperature (i.e., under refrigeration) to, for example, maintain stability.

As used herein, the terms and phrases “foodstuff,” “food supplement,” “beverage,” and “beverage supplement” have the normal meanings for those terms, and are not restricted to pharmaceutical preparations. Other composition forms comprising the thiazolidines prepared by the methods disclosed herein are also included within the present invention. These may, for example, include pure or substantially pure compound such as a foodstuff precursor (such as a rehydratable powder), or a beverage precursor (such as a powder dispersible in water, milk, or other liquid). In some embodiments, the foodstuff, foodstuff supplement, beverage, or beverage supplement is frozen. In some embodiments, the foodstuff, foodstuff supplement, beverage, or beverage supplement is not frozen. The beverage can also be in the form of a slurry where the beverage is a mix of liquid and solid. A beverage or foodstuff is something that is suitable for animal consumption. In some embodiments, the beverage or foodstuff is suitable for human consumption. A composition suitable for animal or human consumption is something that can be ingested without causing harm to the animal or human.

### EXAMPLES

The present invention is now described with reference to the following example. The example is provided for the purpose of illustration only and the invention should in no way be construed as being limited to the example, but rather should be construed to encompass any and all variations which become evident as a result of the teaching provided herein. Those of skill in the art will readily recognize a variety of non-critical parameters that could be changed or modified.

### Example 1

### Preparation of RibCys (Actual Example)

A reactor was purged with nitrogen and kept under nitrogen throughout the entire synthesis. 130.30 kg of USP purified water was added to the reactor. While stifling the reactor, 67.60 kg of D-ribose was added and stirred at room temperature until the solid completely dissolved. 52.50 kg of L-cysteine was added to the reactor. 104.05 kg of ethanol was added to the reactor. The contents of the reactor were stirred at room temperature for about 24 hours. 414.85 kg of ethanol was added to the reactor and stirred for at least 2 hours at a temperature from 18° C. to 20° C. The contents of the reactor were cooled to a temperature less than or equal to 5° C. and held at the temperature of less than 5° C. for at least 2 hours. The precipitated material was centrifuged and washed with ethanol. The product was vacuum dried. The final product had a white color. The synthesis yielded 104.98 kg of RibCys, which was a 95.65% yield and was deemed to be 100% pure as analyzed by HPLC.

What is claimed is:

1. A method of preparing a thiazolidine comprising:
a) contacting a sugar and a cysteine with an aqueous solution under suitable conditions to form the thiazolidine;
b) isolating the thiazolidine; and
c) optionally drying the thiazolidine for a sufficient period of time to yield an anhydrous thiazolidine.

2. The method of claim 1 wherein the sugar is aldose or ketose monosaccharide, wherein said aldose or ketose monosaccharide is glyceraldehyde, glucose, ribose, or fructose.

3. The method of claim 1 wherein the cysteine is free of cystine.

4. The method of claim 1 wherein:
a) the sugar is contacted with the aqueous solution before the cysteine is contacted with the aqueous solution; or
b) wherein the cysteine is contacted with the solution after the sugar is completely dissolved.

5. The method of claim 1 further comprising contacting the solution with an alcohol.

6. The method of claim 5 wherein cysteine and an alcohol are contacted with the solution after the sugar is completely dissolved or the cysteine and an alcohol are contacted with the solution simultaneously.

7. The method of claim 6 wherein the method comprises in the following order:
i) contacting the sugar with the aqueous solution to form a sugar solution;
ii) contacting the cysteine with the sugar solution of i); and
iii) contacting the alcohol with the solution of ii).

8. The method of claim 1 wherein the isolating comprises precipitating the thiazolidine, wherein the thiazolidine is precipitated by contacting the solution with an alcohol and optionally, wherein the precipitated thiazolidine is washed with a washing agent.

9. The method of claim 1 wherein the method yields at least 95% of a calculated theoretical yield and/or the thiazolidine is at least 99% pure as determined by HPLC.

10. The method of claim 1 wherein the thiazolidine comprises less than 225 ppm of an alcohol.

11. The method of claim 1 wherein the isolated thiazolidine comprises a monohydrate thiazolidine.

12. The method of claim 1 wherein the isolated thiazolidine consists of anhydrous thiazolidine.

13. The method of claim 1 wherein the thiazolidine is RibCys, GlcCys, GlycCys, FruCys, GlcNH₂Cys, GlcNHAcCys, and/or any combination thereof.

14. A method of preparing a thiazolidine comprising:
a) contacting an aldose or ketose monosaccharide or an amino sugar, and a cysteine with an aqueous solution under conditions to form the thiazolidine;
b) precipitating the thiazolidine; and
c) vacuum drying the thiazolidine at a temperature that is less than or equal to 65° C.,
wherein the aqueous solution optionally comprises ethanol.

15. The method of claim 14 wherein the thiazolidine is RibCys, GlcCys, GlycCys, FruCys, GlcNH₂Cys, GlcNHAcCys, and/or any combination thereof.

16. A method of preparing a thiazolidine comprising in the following order:
a) contacting an aldose or ketose monosaccharide or an amino sugar with an aqueous solution;
b) contacting the solution of step a) with a cysteine and ethanol;
c) precipitating the solution of step b) to yield a thiazolidine precipitate; and
d) vacuum drying the thiazolidine precipitate.

17. The method of claim 16 wherein step b) comprises in the following order:
i) contacting the solution of step a) with the cysteine; and
ii) contacting the solution of step i) with the ethanol.

18. The method of claim 16 wherein the thiazolidine is RibCys, GlcCys, GlycCys, FruCys, GlcNH₂Cys, GlcNHAcCys, and/or any combination thereof.
